(19) United States
(12) Patent Application Publication
(10) Pub. No.: US 2025/0123023 A1
(43) Pub. Date: Apr. 17, 2025

(54) DEVICE FOR ATTENUATING ULTRAVIOLET RADIATION

(71) Applicant: PATHOGEN REDUCTION SOLUTIONS LIMITED, Horley, Surrey (GB)

(72) Inventors: Robert MCCULLOUGH, Horley, Surrey (GB); Ian SINCLAIR, Horley, Surrey (GB); Julia MARTIN, Horley, Surrey (GB)

(21) Appl. No.: 18/723,139

(22) PCT Filed: Dec. 23, 2022
(86) PCT No.: PCT/GB2022/053390

§ 371 Date: Jun. 21, 2024

(30) Foreign Application Priority Data
Dec. 23, 2021 (GB) 2118916.2

(51) Int. Cl. F24F 13/02; F24F 8/22; F24F 13/08

(52) U.S. Cl. CPC F24F 13/0263; F24F 8/22; F24F 13/0245; F24F 13/08

(57) ABSTRACT

Embodiments described herein relate to a device for attenuating ultraviolet radiation in an air handling system, wherein the device is arranged for insertion in a ducting section of the air handling system, the device comprising: a plurality of elongate flow passages, wherein each of the plurality of elongate flow passages is configured to permit airflow through the elongate flow passage from an inlet of the device to an outlet of the device; wherein each of the elongate flow passages comprises one or more internal walls comprising a coating configured to absorb a proportion of incident ultraviolet radiation.

### FIELD

The present disclosure relates to a device for attenuating ultraviolet (UV) radiation. In particular, the present disclosure relates to a device for attenuating UV radiation within an air handling system. The present disclosure also relates to a method of manufacturing a device for attenuating UV radiation, a method of attenuating electromagnetic radiation and the use of a device for attenuation of electromagnetic radiation.

### BACKGROUND

Air handling systems such as heating, ventilation and air conditioning (HVAC) systems are used to supply air to buildings and other locations. Typically, these air handling systems recirculate recycled air within the building. The recirculation of recycled air can involve recirculation of pathogens, if one or more occupants of the building are infected with a particular disease.

UV radiation (e.g. UVC radiation) can be used to irradiate pathogens present in airflow through an air handling system, thereby damaging the pathogens and rendering them inactive. By using UV radiation to “clean” the airflow in this way, air that is potentially contaminated can be recirculated in the air handling system. An alternative to cleaning the airflow would be to increase the proportion of non-recycled air used in the air handling system. Existing air handling systems are designed to operate with a maximum non-recycled air fraction of approximately 30%. Running these existing systems at 100% non-recycled air would result in insufficient heating or cooling of the air supply, causing discomfort for the building's occupants and potentially resulting in an unusable working environment. Modifying these existing systems to increase the heating or cooling capacity of the air supply systems would incur significant cost, and would result in substantially higher running costs for the building.

Accordingly, using UV radiation to destroy pathogens in recirculated air improves safety for building occupants without requiring increased usage of non-recycled air. By avoiding increased usage of non-recycled air, the significant cost of installing the heating or cooling capacity required for handling increased volumes of non-recycled air is avoided, building running costs are reduced, and energy consumption is reduced.

UV radiation is, however, potentially lethal to humans and other living organisms. The lethality of UV radiation is dependent on the UV dosage that a living organism is exposed to (i.e. UV irradiance multiplied by exposure time). Air handling systems typically require maintenance by human operators. Therefore, air handling systems that incorporate UV sources can represent dangerous working environments for human maintenance workers. In addition, UV radiation can cause degradation of components within air handling systems. For example, UV radiation can damage plastic components such as air flow sensors within air handling systems.

Accordingly, there exists a need for minimising the exposure to UV radiation at locations within an air handling system.

### SUMMARY

This summary introduces concepts that are described in more detail in the detailed description. It should not be used to identify essential features of the claimed subject matter, nor to limit the scope of the claimed subject matter.

According to a first aspect of the present disclosure, there is provided a device for attenuating ultraviolet radiation in an air handling system, wherein the device is arranged for insertion in a ducting section of the handling system, the device comprising: a plurality of elongate flow passages, wherein each of the plurality of elongate flow passages is configured to permit airflow through the elongate flow passage from an inlet of the device to an outlet of the device; wherein each of the elongate flow passages comprises one or more internal walls comprising a coating configured to absorb a proportion of incident ultraviolet radiation.

The device described above allows ultraviolet radiation to be attenuated to a particular level, which may be a safe exposure level for human operators, or a level at which degradation of components within the air handling system is reduced or eliminated. At the same time, the use of elongate flow passages allows air to flow through the device. The device therefore attenuates ultraviolet radiation while minimising impedance to air flow through the ducting section.

The device may be arranged for insertion in the ducting section such that any airflow through the ducting section flows through the elongate flow passages of the device. In this way, any air or radiation can only pass through the ducting section via an elongate flow passage.

The plurality of elongate flow passages may be arranged such that, collectively, the plurality of elongate flow passages has a substantially square or rectangular cross-section. This allows the elongate flow passages to be easily mounted in an air handing system that includes ducts with similar cross-sections.

Each of the plurality of elongate flow passages may be configured to permit air to flow substantially unimpeded through the elongate flow passage from the inlet to the outlet. Minimising the impedance to air flowing through the ducting section reduces the pressure drop in the air handling system resulting from including the device in the ducting section.

Each of the plurality of elongate flow passages may be straight. This minimises the resistance to air flowing through the elongate flow passage from the inlet to the outlet.

In turn, this minimises the pressure drop within the air handling system as a result of implementing the device in the ducting section.

Each of the elongate flow passages may have a length that is significantly greater than a diameter of the flow passage. For example, the length may be four or more times greater than the diameter.

Each of the plurality of elongate flow passages may have an aspect ratio calculated by dividing a length of the elongate flow passage by a diameter of the elongate flow passage. The aspect ratio of each of the plurality of elongate flow passages may be greater than or equal to 4. An aspect ratio of greater than or equal to 4 means that ultraviolet photons are likely to reflect off the internal walls of the elongate flow passage. In addition, an aspect ratio of greater than or equal to 4 provides a low range of angles over which a photon can pass through the elongate flow passage without colliding with the internal walls.

The aspect ratio of each of the plurality of elongate flow passages may be less than or equal to 50. An aspect ratio of less than 50 reduces the boundary layer effects through each of the elongate flow passages, thereby reducing the pressure drop resulting from implementation of the device in the ducting section.

The aspect ratio of each of the plurality of elongate flow passages may be less than or equal to 20. An aspect ratio of less than 20 further reduces the boundary layer effects through each of the elongate flow passages, thereby further reducing the pressure drop resulting from implementation of the ducting section.

The coating may be configured to absorb a proportion of incident ultraviolet-C (UVC) radiation. More specifically, the coating may be configured to absorb a proportion of incident UVC radiation having a wavelength of between about 200 nm and 280 nm, more preferably between 210 nm and 260 nm, and even more preferably about 222 nm or about 254 nm.

The coating may be configured to reflect less than 60% of incident UVC radiation, preferably less than 50% of incident UVC radiation, more preferably less than 40% of incident UVC radiation, more preferably less than 30% of incident UVC radiation, more preferably less than 20% of incident UVC radiation, more preferably less than 10% of incident UVC radiation, and most preferably less than 5% of incident UVC radiation. The proportions of reflected UVC radiation given in the foregoing list provide progressively lower amounts of radiation reflected by the coating on the internal walls of the elongate flow passages, thereby providing progressively higher attenuations of ultraviolet radiation.

The coating may comprise one or more coats of black paint. More preferably, the coating may comprise two or more coats of black paint, and more preferably three or more coats of black paint. A black surface absorbs incident ultraviolet radiation, thereby attenuating the ultraviolet radiation. Using two or more coats of black paint provides increased attenuation of ultraviolet radiation when compared with a single coat of black paint. Attenuation is further increased by using three or more coats of black paint.

The one or more walls of each of the plurality of elongate flow passages may be formed of aluminium. Aluminium is not degraded by ultraviolet radiation, meaning that the elongate flow passages are not damaged by exposure to the ultraviolet radiation.

Each of the plurality of elongate flow passages may have a hexagonal cross-section. Using a hexagonal cross-section allows the elongate flow passages to be easily fabricated using a honeycomb structure.

The plurality of elongate flow passages may be defined by a honeycomb structure of the device. The honeycomb structure helps to damp out turbulence in the airflow, which can provide beneficial effects downstream, such as reduced mixing and reduced frictional pressure drop.

The device may be for attenuating ultraviolet radiation in a heating, ventilation and air conditioning, HVAC, system comprising a source of ultraviolet radiation. The device may be arranged for insertion in a ducting section of the HVAC system. Accordingly, the device may be used with existing building infrastructure in order to attenuate ultraviolet radiation that is being used to inactivate pathogens within the airflow through the HVAC system.

According to a second aspect of the present disclosure, there is provided a ducting section comprising a device according to the first aspect.

The ducting section may be for use in a heating, ventilation and air conditioning, HVAC, system comprising a source of ultraviolet radiation.

The ducting section may further comprise one or more walls; and a removable casing, wherein the removable casing is arranged to cover an opening in the one or more walls. Providing a removable casing arranged to cover an opening in the one or more walls of the ducting section allows access to the interior of the ducting section, for example, for maintenance of the device.

The opening may be arranged to allow removal of the device from the ducting section. Permitting removal of the device from the ducting section allows the device to be maintained, removed, and/or replaced.

The device may be attachable to the removable casing. For example, the device may be removably attached to the removable casing, or permanently attached to the removable casing. Attaching the device to the removable casing improves the ease of removing the device from the ducting section, because the device can be removed from the exterior of the ducting section.

According to a third aspect of the present disclosure, there is provided a system, comprising: a ducting section according to the second aspect; and one or more baffles arranged between the plurality of elongate flow passages and an ultraviolet radiation source, wherein the one or more baffles are configured to prevent photons emitted by the ultraviolet radiation source from entering the plurality of elongate flow passages in a direction parallel to the plurality of elongate flow passages.

The baffles prevent ultraviolet photons from passing directly through the ducting section without colliding with the internal walls of the elongate flow passages. Accordingly, the baffles ensure that all ultraviolet rays collide with the internal walls, meaning that all ultraviolet rays are attenuated by the ducting section.

The system may comprise the source of ultraviolet radiation. The system may comprise: a first ducting section according to the second aspect, wherein the first ducting section is disposed upstream of the source of ultraviolet radiation; and a second ducting section according to the second aspect, wherein the second ducting section is disposed downstream of the ultraviolet radiation source.

According to a fourth aspect of the present disclosure, there is provided a system, comprising: a source of ultraviolet radiation; a first ducting section according to the second aspect, wherein the first ducting section is disposed upstream of the source of ultraviolet radiation; and a second ducting section according to the second aspect, wherein the second ducting section is disposed downstream of the ultraviolet radiation source.

According to a fifth aspect of the present disclosure, there is provided a method of manufacturing a device for attenuating ultraviolet radiation, the method comprising: providing a device comprising a plurality of elongate flow passages, wherein each of the plurality of elongate flow passages is configured to permit airflow through the elongate flow passage from an inlet of the device to an outlet of the device; and applying a first coat of a coating configured to absorb a proportion of incident ultraviolet radiation to one or more internal walls of each of the plurality of elongate flow passages. The method may comprise applying a second coat of the coating to the one or more internal walls of each of the plurality of elongate flow passages. The coating may comprise black paint. Providing the device comprising the plurality of elongate flow passages may comprise providing the device with elongate flow passages, each having an aspect ratio calculated by dividing a length of the elongate flow passage by a diameter of the elongate flow passage. The aspect ratio of each of the plurality of elongate flow passages may be greater than or equal to 4, optionally less than or equal to 50, and preferably less than or equal to 20.

According to a sixth aspect of the present disclosure, there is provided a method of attenuating electromagnetic radiation, the method comprising: providing a device comprising a plurality of elongate flow passages, wherein each of the plurality of elongate flow passages is configured to permit airflow through the elongate flow passage from an inlet of the device to an outlet of the device; and disposing the device between a source of the electromagnetic radiation and a particular location, thereby reducing the amount of electromagnetic radiation that reaches the particular location.

The device allows electromagnetic radiation to be attenuated to a particular level. At the same time, the use of elongate flow passages allows air to flow through the device. The device therefore attenuates electromagnetic radiation while minimising impedance to air flow. The electromagnetic radiation emitted by the source or electromagnetic radiation may be ultraviolet radiation. The ultraviolet radiation may be UVC radiation. The device may be a device according to the first aspect.

According to a seventh aspect of the present disclosure, there is provided the use of a device comprising a plurality of elongate flow passages for attenuation of electromagnetic radiation, wherein each of the plurality of elongate flow passages is configured to permit airflow through the elongate flow passage from an inlet of the device to an outlet of the device. The device allows electromagnetic radiation to be attenuated to a particular level. At the same time, the use of elongate flow passages allows air to flow through the device. The device therefore attenuates electromagnetic radiation while minimising impedance to air flow. The electromagnetic radiation may be ultraviolet radiation. The ultraviolet radiation may be UVC radiation. The device may be a device according to the first aspect.

According to an eighth aspect of the present disclosure, there is provided a heating, ventilation and air conditioning, HVAC, system comprising: a source of ultraviolet radiation; and a ducting section according to the second aspect.

### BRIEF DESCRIPTION OF FIGURES

Specific embodiments are described below by way of example only and with reference to the accompanying drawings, in which:

FIG. 1 is a front view of a device for attenuating ultraviolet radiation disposed within a ducting section.

FIG. 2 is a cross-section through line A-A in FIG. 1.

FIG. 3 is a perspective view of a single elongate flow passage of the device shown in FIG. 1, in isolation from the other elongate flow passages of the device.

FIG. 4 is a schematic diagram of an air handling system comprising a device for attenuating ultraviolet radiation.

FIG. 5 is a schematic diagram of an air handling system comprising a device for attenuating ultraviolet radiation and two baffles.

FIG. 6 is a schematic diagram of an air handling system comprising two devices for attenuating ultraviolet radiation.

FIG. 7 is a schematic diagram of a ducting section comprising a device for attenuating ultraviolet radiation.

FIG. 8 is a flowchart of a method of manufacturing a device for attenuating radiation.

FIG. 9 is a flowchart of a method of attenuating electromagnetic radiation.

FIG. 10 is a diagram showing locations of ultraviolet irradiance measurements in a system comprising a device for attenuating ultraviolet radiation.

FIG. 11 is a plot showing how ultraviolet irradiance varies with distance from an outlet of a device for attenuating ultraviolet radiation, wherein internal walls of the device are not coated.

FIG. 12 is a plot showing how ultraviolet irradiance varies with distance from an outlet of a device for attenuating ultraviolet radiation, wherein internal walls of the device are coated with black paint.

FIG. 13 is a plot showing the ultraviolet irradiance at different measurement locations for a device having internal walls coated with black paint and a device having internal walls coated with two coats of black paint.

### DETAILED DESCRIPTION

Implementations of the present disclosure are explained below with particular reference to preventing leakage of UVC photons within an air handling system such as a heating, ventilation, and air conditioning (HVAC) system. It will be appreciated, however, that the implementations described herein are applicable to preventing leakage of UVC photons in other systems, in which there is a risk of exposure from UVC photons (e.g. through ducting or conduits). It will further be appreciated that the implementations described herein are not limited to preventing leakage of UVC photons, but may be used to prevent leakage of photons over the entire electromagnetic spectrum.

In general terms, therefore, the interposition of a device as disclosed herein between a source of electromagnetic radiation and a particular location reduces the number of photons that reach the particular location, while permitting fluid flow between the source of electromagnetic radiation and the particular location. This is achieved through absorption of photon energies within the device, while permitting fluid flow through the device.

As one specific example, the interposition of a device as disclosed herein between a source of UVC photons (e.g. a UVC lamp) within a HVAC system and a maintenance location within the HVAC system reduces the number of photons that reach the particular location, while allowing air to flow from the source of UVC photons to the maintenance location. A device as disclosed herein may, therefore, be used to reduce the level of UVC radiation in an air handling system to within an acceptable exposure range for human maintenance workers.

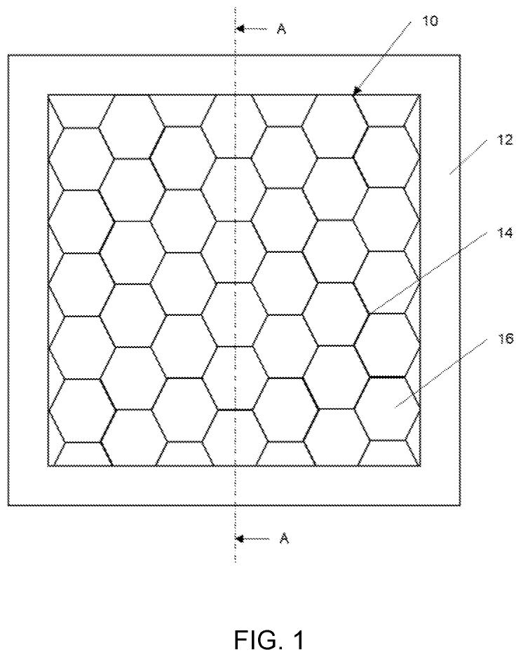

FIG. 1 is a front view of a device 10, which may be mounted in an air handling system such as a heating, ventilation, and air conditioning (HVAC) system. In the example shown in FIG. 1, the device 10 is disposed within a ducting section 12 having a square cross-section. The device 10 may be installed within an existing ducting section of an air handling system. Alternatively, the device 10 may be provided as part of a ducting section for an air handling system. In the latter case, the device 10 may be installed in the air handling system by replacing an existing ducting section of the air handling system with ducting section that comprises the device 10.

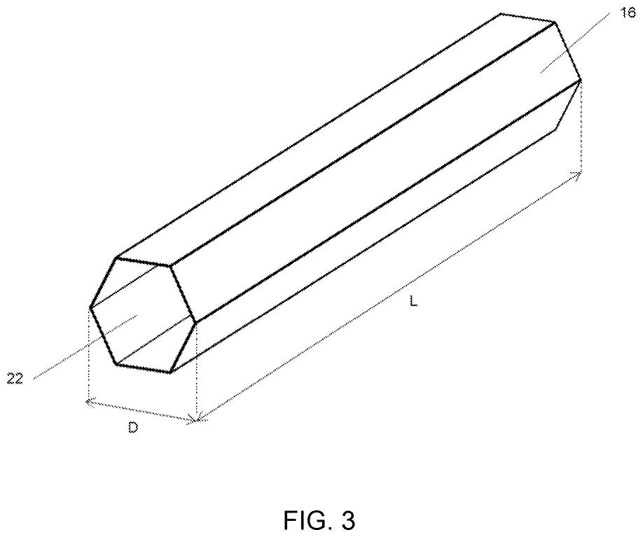

The device 10 comprises a honeycomb structure 14 that defines a plurality of elongate flow passages 16. As shown in FIGS. 1 and 3, the individual elongate flow passages 16 have a hexagonal cross-section. The honeycomb structure 14 allows air to pass through the device 10 (i.e. through the elongate flow passages 16). The honeycomb structure 14 helps to damp out turbulence in the airflow, which can provide beneficial effects downstream, such as reduced mixing and reduced frictional pressure drop. The honeycomb structure 14 may, for example, be a Hexweb® aluminium honeycomb available from Hexcel Corporation of Stamford, CT, USA. Such honeycomb is available with wall thicknesses of between 0.0007 inches (0.3 mm) and 0.004 inches (1.6 mm).

The honeycomb structure 14 is formed of a material that is not degraded by ultraviolet radiation, so that the device 10 is not damaged by exposure to an ultraviolet radiation source. For example, the honeycomb structure 14 may be formed of aluminium.

The honeycomb structure 14 has a substantially square or rectangular cross-section, allowing it to be disposed within a square or rectangular cross-section of a ducting section (e.g. the square cross-section of the ducting section 12 shown in FIG. 1). This means that, collectively, the plurality of elongate flow passages 16 has a substantially square or rectangular cross-section. The edges of the honeycomb structure 14 will, of course, be non-straight, owing to the tessellation of the hexagonal elongate flow passages 16. Accordingly, the cross-section of the honeycomb structure 14 will not be a strict square or rectangle shape. In this context, therefore, “substantially square or rectangular” means that the overall envelope defined by the corners of the honeycomb structure 14 is square or rectangular, without requiring the edges of the honeycomb structure 14 to be straight.

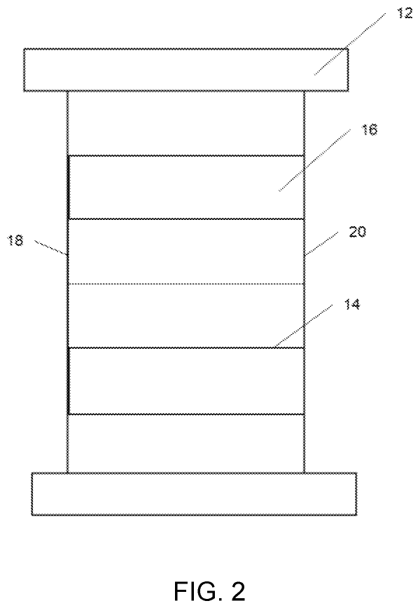

As shown in FIG. 2, the honeycomb structure 14 comprises an inlet face 18 and an outlet face 20. The inlet face 18 defines an inlet to the device 10, while the outlet face 20 defines an outlet from the device 10. More specifically, the inlet face 18 defines an inlet to each of the elongate flow passages 16, while the outlet face defines an outlet from each of the elongate flow passages 16. Each elongate flow passage 16 of the honeycomb structure 14 allows air to flow from the inlet to the device 10 to the outlet from the device 10 (i.e. from the inlet face 18 to the outlet face 20). In particular, the honeycomb structure 14 fills an internal volume defined by the ducting section 12, such that any air flowing through the ducting section 12 passes through an elongate flow passage 16.

Each elongate flow passage 16 shown in FIG. 2 is straight. This minimises the resistance to air flowing through the elongate flow passage 16 from the inlet face 18 to the outlet face 20. In turn, this minimises the pressure drop within the air handling system as a result of implementing the device 10. Each elongate flow passage 16 is also configured to permit air to flow substantially unimpeded through the elongate flow passage 16 from the inlet face 18 to the outlet face 20. Each elongate flow passage 16 will, of course, cause some impediment to air flow through the elongate flow passage 16 (as a result of the boundary layer at the surface of the internal walls 22 (shown in FIG. 3) of the elongate flow passage 16). In this context, therefore, “substantially unimpeded” means that nothing protrudes from the internal walls 22 towards the centre of the elongate flow passage 16, and the internal walls 22 themselves are not curved or angled in the direction of airflow, while recognising that boundary layer effects will be present within the elongate flow passage 16.

FIG. 3 shows an individual elongate flow passage 16 of the device 10 shown in FIGS. 1 and 2. FIG. 3 shows the hexagonal cross-section of the elongate flow passage 16. As shown schematically in FIG. 3, the length of each elongate flow passage 16 is significantly greater than the diameter of the elongate flow passage 16. In this context, “significantly greater” is to be interpreted as four or more times greater.

As shown in FIG. 3, each elongate flow passage 16 comprises one or more internal walls 22. Specifically, in the example of an elongate flow passage 16 with a hexagonal cross-section, each elongate flow passage 16 has six internal walls 22. The internal walls 22 of the elongate flow passage 16 are configured to attenuate ultraviolet radiation that is incident on the internal walls 22.

A desired level of attenuation of the ultraviolet radiation can be achieved by tuning a number of parameters of the elongate flow passages 16. For example, the desired level of attenuation may be to below the RG2 (Risk Group 2) actinic UV limit defined in standards IEC 62471:2006 and BS EN 62471:2008 (Photobiological safety of lamps and lamp systems). The RG2 actinic UV limit is 0.03 W/m². Specifically, a desired level of attenuation can be achieved by varying one or more of: (i) an aspect ratio of the elongate flow passage 16; (ii) the nature of the reflection of incident radiation from the internal walls 22; and (iii) the reflectivity of the internal walls 22 to ultraviolet radiation. The effect of varying these parameters is explained in the following paragraphs.

The aspect ratio of an elongate flow passage 16 is defined as the ratio of the length (L in FIG. 3) of the elongate flow passage 16 to the diameter (D in FIG. 3) of the elongate flow passage 16. In other words, the aspect ratio may be calculated by dividing the length of the elongate flow passage 16 by the diameter of the elongate flow passage 16.

Although the streamlines of air flowing in the device 10 are parallel to the internal walls 22, optical radiation enters the honeycomb structure 14 at a variety of angles. Radiation that enters the honeycomb structure 14 normally (i.e. parallel to the elongate flow passages 16) will pass through relatively unimpeded. However, radiation that enters at oblique angles (above a certain threshold determined by the aspect ratio) will collide with the internal walls 22 at least once.

For photons that collide with the internal walls 22 of the elongate flow passage 16 at a first aspect ratio (e.g. 4:1), increasing the aspect ratio (e.g. to 10:1) increases the number of reflections of the photon within the elongate flow passage 16. In addition, increasing the aspect ratio of the elongate flow passage 16 reduces the range of angles over which a photon can pass through the elongate flow passage 16 without colliding with the internal walls 22.

The number of reflections of a photon increases with the obliqueness of the incident angle. For example, an elongate flow passage 16 with an aspect ratio of 10:1 will allow photon rays at incident angles of approximately +/−5.7 degrees from normal to pass through the elongate flow passage 16 without colliding with the internal walls 22. Rays at incident angles from 5.7 degrees to about 16 degrees from normal will pass through the elongate flow passage 16 with a single reflection off the internal walls 22. Rays at incident angles from about 16 degrees to about 29 degrees will pass through the elongate flow passage 16 with two reflections off the internal walls 22.

The above discussion is applicable to the case of specular reflection. When radiation is reflected by specular reflection, a ray is reflected at an equal but opposite angle to the angle of incidence. For example, if a ray of ultraviolet photons collides with an internal wall 22 at an incidence angle of 20 degrees to the internal wall 22, measured between the incident ray and the internal wall 22 before the collision point, then the ray is reflected at a reflected angle of 20 degrees to the internal wall 22 in the opposite direction (that is, measured between the reflected ray and the internal wall 22 after the collision point). Specular reflection would occur, for example, if the internal walls 22 were bare aluminium. For UVC radiation, the reflectivity of bare aluminium is approximately 70%, meaning that rays with two collisions would be attenuated by about half. The level of attenuation would be higher for oblique rays greater than 29 degrees.

It will be clear from the above discussion that increasing the aspect ratio increases the level of attenuation of ultraviolet radiation. However, very high aspect ratios would create large boundary layer effects through each of the elongate flow passages 16, resulting in a pressure drop. For this reason, the aspect ratio is preferably less than or equal to 50:1. More preferably, the aspect ratio is less than or equal to 20:1, in order to further reduce the pressure drop resulting from usage of the device 10.

A further downside to high aspect ratios is that the device 10 would occupy a large amount of space within the air handling system. An aspect ratio of less than or equal to 50:1 is also preferred in order to minimise the volume taken up by the device 10, and an aspect ratio of less than or equal to 20:1 is more preferred in order to further minimise the volume of the device 10.

One way of minimising the volume of the device 10 would be to reduce the absolute diameter of the elongate flow passages 16. For example, halving the diameter of an elongate flow passage 16 means that the length of an elongate flow passage 16 can also be halved, in order to achieve a given aspect ratio.

However, using smaller diameter elongate flow passages 16 means that the device 10 comprises a greater number of elongate flow passages 16. In other words, more elongate flow passages 16 are required in order to fill the cross-sectional area of the ducting section 12. The internal walls 22 have a given thickness (e.g. between 0.3 mm and 1.6 mm for a honeycomb structure 14 formed from Hexweb® aluminium honeycomb). Therefore, increasing the number of elongate flow passages 16 increases the proportion of the cross-section of the device 10 that is occupied by the honeycomb structure 14 itself (and correspondingly reduces the open area of the device 10 provided by the elongate flow passages).

Reducing the open area of the device 10 impedes air flow through the device 10, thereby increasing the pressure drop through the device 10. It is therefore preferable for the elongate flow passages 16 to have a diameter of between 6 mm and 12 mm. This range also corresponds to the ranges of honeycomb cell sizes of honeycomb structures formed from Hexweb® aluminium honeycomb.

The above discussion regarding increasing the number of collisions by increasing the aspect ratio assumes that the reflection off the internal walls 22 of the elongate flow passages 16 is specular reflection. The number of collisions within an elongate flow passage 16 can be increased by making the reflection off the internal walls 22 more diffuse (i.e. where the incident radiation is scattered in a range of directions). The energy of the reflected ray is reduced with each collision (e.g. a 30% reduction per collision for bare aluminium), so increasing the number of collisions by promoting diffuse reflection reduces the energy of rays at the outlet face 20 of the device 10. This means that the ultraviolet radiation can be attenuated by promoting diffuse reflection off the internal walls 22.

The reflection can be made more diffuse by applying a coating to the material used to fabricate the honeycomb structure 14 (e.g. aluminium). As one example, the aluminium material of the honeycomb structure 14 may be coated with black paint (e.g. flat black automotive paint), which reflects incident radiation in a more diffuse manner (i.e. a less specular manner) than bare aluminium. This means that incident radiation can be reflected in an increased range of directions, including back to where it originated. Multiple layers of paint (e.g. at least two coats, or at least three coats) may be used in order to further increase the diffuse nature of the internal walls 22. The black paint may be, for example, Halfords® Matt Black Car Paint, available from Halfords Group Plc, Redditch, UK. This paint reflects between 0% and 5% of incident radiation at ultraviolet wavelengths, as reported in “Common Black Coatings-Reflectance and Ageing Characteristics in the 0.32 μm to 14.3 μm Wavelength Range”, Dury et al, Optics Communications, 270 (2): 262-272, February 2007, the contents of which are hereby incorporated by reference.

By implementing internal walls 22 that reflect incident radiation in a number of directions (i.e. in a diffuse manner), the number of collisions of a ray of ultraviolet radiation with the internal walls 22 is increased, when compared with specular reflection off the internal walls 22. A coated surface of the internal walls 22 can, therefore, be implemented in conjunction with a smaller aspect ratio, in order to achieve a given attenuation of ultraviolet radiation. This allows the device 10 to be more compact.

The above discussions concerning aspect ratio and the nature of the reflection of incident radiation (i.e. diffuse or specular) assume that a certain percentage of radiation is reflected by the internal walls 22. For example, bare aluminium reflects approximately 70% of incident UVC radiation. In order to reduce the energy of the reflected ray, the internal walls 22 can be configured to reflect a lower proportion of incident UVC radiation. In other words, the internal walls 22 can be configured to absorb a higher proportion of incident UVC radiation. By absorbing a higher proportion of incident UVC radiation, the energy of the rays at the outlet 20 of the ducting section is reduced. This means that the ultraviolet radiation can be attenuated by reflecting a lower proportion of incident radiation.

The reflectivity of the internal walls 22 to incident UVC radiation may be reduced by fabricating the honeycomb structure 14 from a material that is less reflective to UVC radiation, or by adding a non-reflective (or less reflective) coating to the material. As one example, the aluminium material of the honeycomb structure 14 may be coated with black paint, which absorbs incident radiation. This means that the energy reflected ray (which is also reflected by diffuse reflection, as described above) is lower. Multiple layers of paint may be used in order to further reduce the reflectivity to UVC radiation.

By configuring the internal walls 22 to be more reflect a lower proportion of incident UVC radiation than, for example, bare aluminium, the energy of reflected UVC radiation is reduced. This means that internal walls 22 that are less reflective to UVC radiation can be used in conjunction with a smaller aspect ratio, in order to achieve a given attenuation of ultraviolet radiation. This allows the device 10 to be more compact. In order to reduce the energy of reflected radiation, the reflectivity of the internal walls 22 is preferably less than 60% (which provides an improvement over bare aluminium), and more preferably less than 20%, which provides improved performance (i.e. higher attenuation). A reflectivity of about 10% or less (e.g. as provided by flat black automotive paint) can provide further improved performance. A yet further improvement in attenuation reduction can be achieved by configuring the internal walls with a reflectivity of less than 5%.

The following table shows the effect on percentage reductions in ultraviolet irradiance at different aspect ratios, for (i) a device having elongate flow passages with internal walls coated with a coat of matte black paint, resulting in a reflectivity to ultraviolet radiation of approximately 1-5%; and (ii) a device having elongate flow passages with uncoated internal walls (i.e. bare aluminium walls). The pressure drop resulting from implementation of the device is also shown, for an air flow of 6 m/s.

| | Reduction in irradiance (%) | | Pressure |
|---|---|---|---|
| | Painted internal | Bare aluminium | drop |
| Aspect ratio | walls | internal walls | (Pa) |
| 5 | 99% | 88% | 18 |
| 10 | 99.75% | 96% | 30 |
| 20 | 99.95% | 99% | 50 |

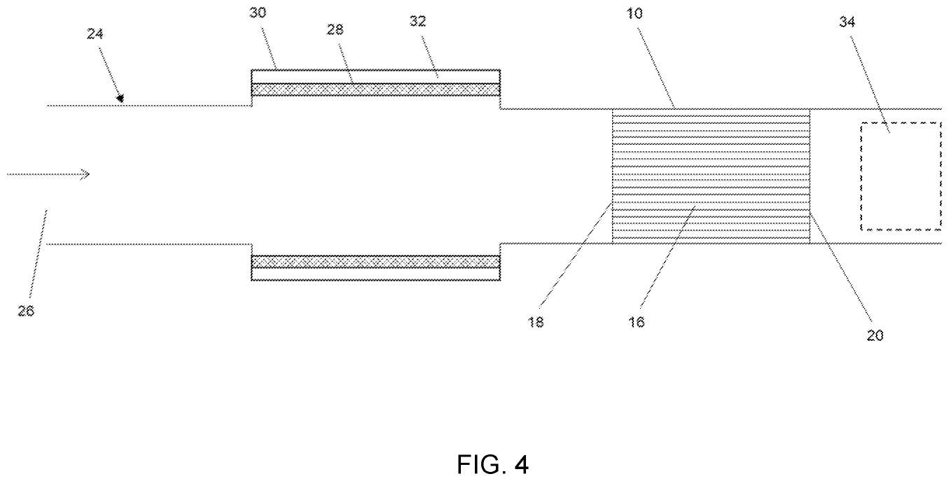

FIG. 4 is a schematic diagram showing the device 10 implemented in an air handling system 24. The air handling system 24 comprises an inlet 26. The inlet 26 may receive a proportion of air that is recirculated from within a building in which the air handling system 24 is implemented. The direction of airflow is indicated by the arrow in FIG. 4. At least one ultraviolet radiation source (shown in FIG. 4 as ultraviolet lamps 28) is located downstream of the inlet 26. In the example shown in FIG. 4, the ultraviolet lamps 28 are housed in an air cleaning section 30. The air cleaning section 30 includes recesses 32 that house the ultraviolet lamps 28, in order to mitigate obstruction of airflow within the air cleaning section 30. The ultraviolet lamps 28 provide an energy flux density within the air cleaning section 30, which kills any pathogens in the air flowing through the air cleaning section 30.

The device 10 is located downstream of the air cleaning section 30. Specifically, the device 10 is located between the air cleaning section 30 and a maintenance location 34 within the air handling system 24. The device 10 attenuates the ultraviolet radiation, meaning that the irradiance at the outlet 20 from the device 10 is lower than the irradiance at the inlet 18 to the device 10. In one example, the device 10 is configured to attenuate the ultraviolet radiation to a level that is safe for a human maintenance worker to carry out maintenance at the maintenance location 34. In another example, components subject to degradation by ultraviolet radiation are located downstream of the device 10, and the device 10 is configured to attenuate the ultraviolet radiation to a level that prevents or slows down degradation of those components.

As the ultraviolet lamps 28 are housed in recesses 32 of the air cleaning section 30, none of the rays from the ultraviolet lamps 28 enters the device 10 in a direction that is parallel to the elongate flow passages 16. Using parameters relating to the positioning of the ultraviolet lamps 28 within the recesses 32 and the distance between the ultraviolet lamps 28 and the inlet face 18 of the device 10, the minimum incidence angle of ultraviolet rays can be calculated. Calculating the minimum incidence angle allows the aspect ratio of the elongate flow passages 16 to be tailored so that ultraviolet rays are unable to pass through the elongate flow passages 16 without colliding with the internal walls 22. In one example, the aspect ratio can be tailored to ensure that ultraviolet rays at the minimum incidence angle collide with the internal walls 22 at least once.

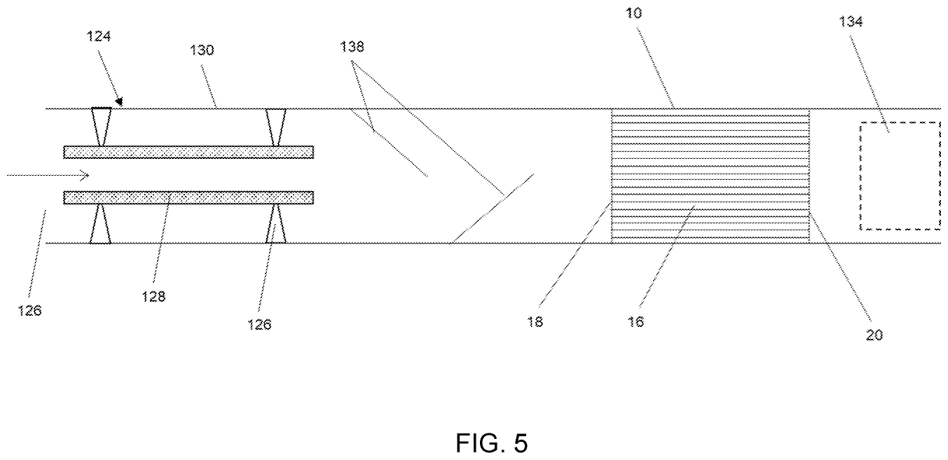

FIG. 5 is a schematic diagram showing the device 10 implemented in an alternative air handling system 124. As with the air handling system 124 shown in FIG. 4, the air handling system 124 comprises an inlet 126, with the airflow direction indicated by the arrow in FIG. 5. The air handling system 124 also comprises an air cleaning section 130. However, in contrast to the air cleaning section 130 shown in FIG. 4, the air cleaning section 130 comprises an ultraviolet radiation source (shown in FIG. 5 as ultraviolet lamps 128) disposed within the airflow through the air cleaning section 130. In particular, the ultraviolet lamps 128 are mounted on supports 136 that protrude from the internal walls of the air cleaning section 130.

The device 10 is located downstream of the air cleaning section 130. Specifically, the device 10 is located between the air cleaning section 130 and a maintenance location 134 within the air handling system 124. The device 10 attenuates the ultraviolet radiation, meaning that the irradiance at the outlet 20 from the device 10 is lower than the irradiance at the inlet 18 to the device 10.

As the ultraviolet lamps 128 are disposed within the airflow through the air handling system 124, ultraviolet rays could enter the device 10 parallel to the elongate flow passages 16 if there were no obstruction between the ultraviolet lamps 128 and the device 10. Ultraviolet rays entering the device 10 parallel to the elongate flow passages 16 can pass directly through the device 10 without colliding with the internal walls 22. For such ultraviolet rays, the device 10 does not reduce the energy of the rays, meaning that these rays could be potentially harmful for maintenance workers at the maintenance location 134.

To prevent ultraviolet rays from entering the device 10 parallel to the elongate flow passages 16, one or more baffles 138 are disposed between the ultraviolet lamps 128 and the device 10. In the example shown in FIG. 5, two baffles 138 are shown. The baffles 138 divert the air flowing through the air handling system 124. The baffles 138 are orientated so as to prevent a direct line-of-sight between the ultraviolet lamps 128 and the outlet face 20 of the device 10. This means that ultraviolet rays emitted by the ultraviolet lamps 128 are unable to pass through an elongate flow passage 16 in a direction that is parallel to the elongate flow passage 16.

Although two baffles 138 are shown in FIG. 5, it will be appreciated that the direct line-of-sight between the ultraviolet lamps 128 and the outlet face 20 may be prevented by using a single baffle. The direct line-of-sight may, alternatively, be prevented in other ways. As one example, a corner ducting section (e.g. 90 degree corner section, 90 degree curved section, or other angled section) may be disposed between the ultraviolet lamps 128 and the device 10.

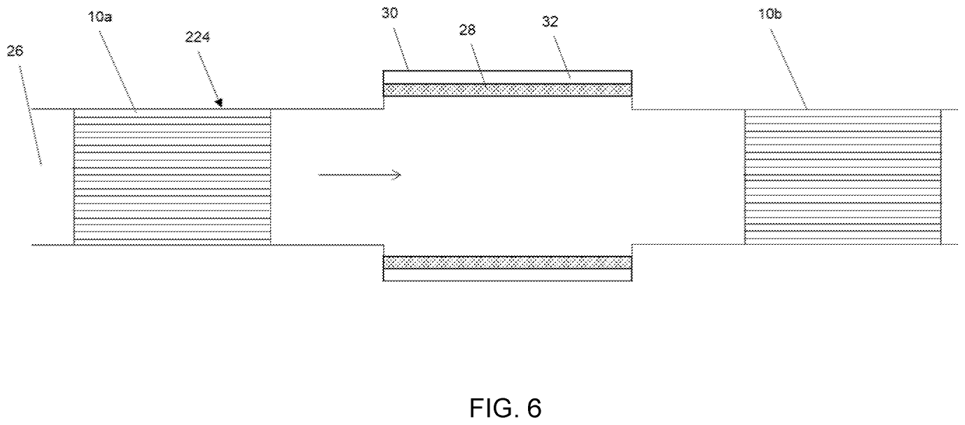

FIG. 6 is a schematic diagram showing a further alternative handling system 224, in which two devices 10 are implemented. The air cleaning system 224 includes the air cleaning section 30 shown in FIG. 4. The devices 10 are indicated in FIG. 6 as a first device 10a, located upstream of the air cleaning section 30, and a second device 10b, located downstream of the air cleaning section 30. Although not shown in FIG. 6, a first maintenance location may be located upstream of the first device 10a, and a second maintenance location may be located downstream of the second device 10b. The first device 10a is configured to attenuate ultraviolet radiation from the ultraviolet lamps 28 in the air cleaning section 30, meaning that the ultraviolet irradiance upstream of the first device 10a is lower than the irradiance within the air cleaning section 30. Likewise, the second device 10b is also configured to attenuate ultraviolet radiation from the ultraviolet lamps 28 in the air cleaning section 30, meaning that the ultraviolet irradiance downstream of the second device 10b is lower than the irradiance within the air cleaning section 30.

It will be appreciated that multiple devices 10 may also be used in conjunction with the air cleaning section 130 shown in FIG. 5 (optionally in combination with multiple baffles 138). In other words, the use of multiple devices 10 is not limited to air handling systems that incorporate the air cleaning section 30 shown in FIGS. 4 and 6.

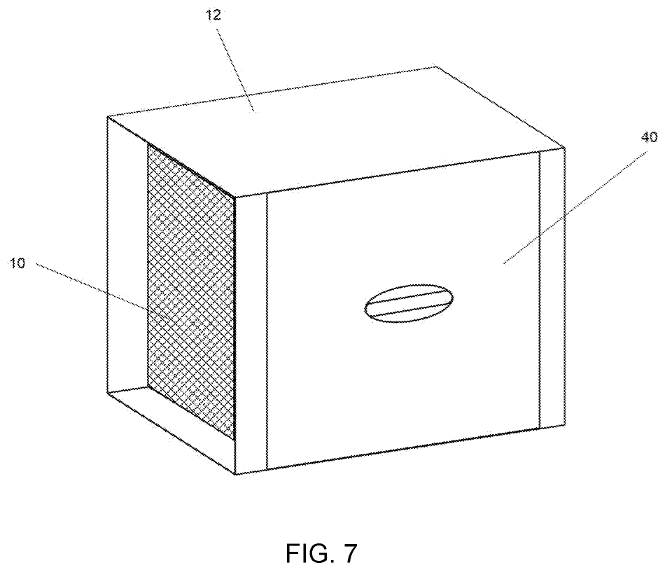

FIG. 7 is a schematic diagram of a ducting section 12 in which the device 10 described with reference to FIGS. 1 to 6 is disposed. The ducting section 12 may, for example, comprise the device 10. As shown in FIG. 7, the ducting section 12 comprises a removable casing 40 that covers an aperture (not shown) in a wall of the ducting section 12. The removable casing 40 may be removed in order to allow access to the device 10 (e.g. for maintenance). The aperture in the ducting section 12 may be large enough to permit the device 10 to be removed from the ducting section 12. In particular, the aperture may have a height that is greater than the height of the device 10, and a width that is greater than the length of the device 10. By allowing removal of the device 10 from the ducting section 12, the device 10 can be removed for maintenance or replacement, for example.

In one particular implementation, the device 10 may be attachable to the removable casing 40 (e.g. removably or permanently attached). This allows an operator to slide the device 10 out of the ducting section 12 using the removable casing 40. Therefore, the device 10 can be removed in a simple manner, without requiring the operator to access the interior of the ducting section 12.

The ducting section 12 may be for use in a HVAC system comprising a source of ultraviolet radiation such as one or more ultraviolet lamps.

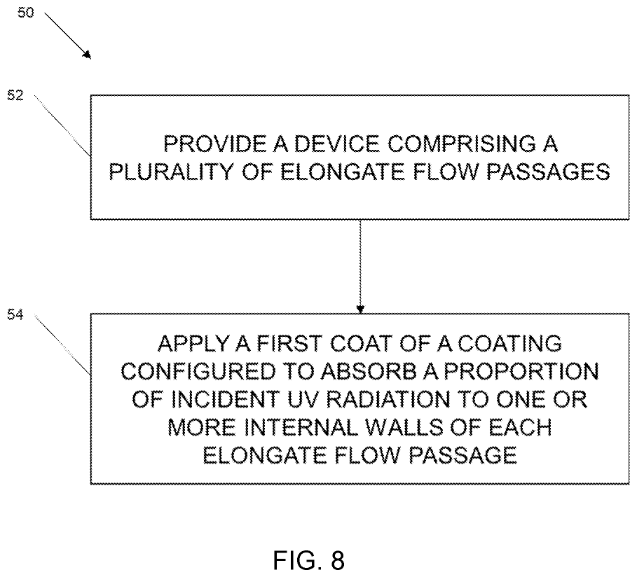

FIG. 8 is a flowchart of a method 50 of manufacturing a device for attenuating ultraviolet radiation. At 52, a device comprising a plurality of elongate flow passages is provided. Each of the plurality of elongate flow passages is configured to permit airflow through the elongate flow passage from an inlet of the device to an outlet of the device. For example, the device may comprise a honeycomb structure that provides the plurality of elongate flow passages. Providing the device may comprise comprising the plurality of elongate flow passages may comprise selecting an aspect ratio of each of the elongate flow passages. The aspect ratio of each elongate flow passage may be defined as the length of the elongate flow passage divided by the diameter of the elongate flow passage. For example, an aspect ratio of between four and 50 (and preferably less than 20) may be selected.

At 54, a first coat of a coating configured to absorb a proportion of incident ultraviolet radiation is applied to one or more internal walls of each elongate flow passage. The coating may comprise black paint (e.g. matte black paint). As an optional further step, the method may comprise applying one or more further coats (i.e. a second coat and an optional third coat and further optional additional coats) to the one or more internal walls of each elongate flow passage. Accordingly, the device 10 described with reference FIGS. 1 to 6 may be produced by the method 50.

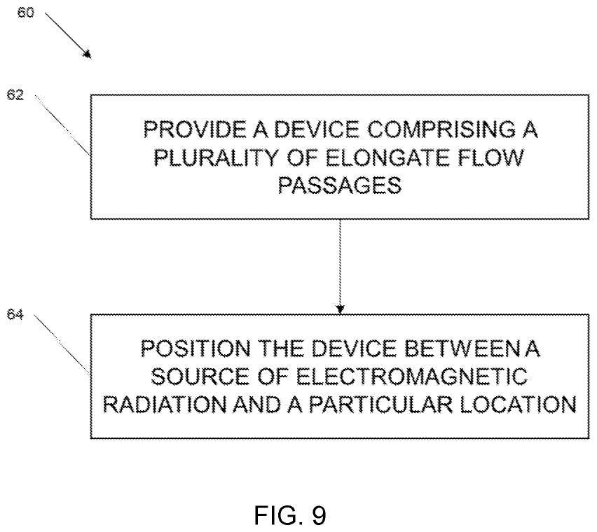

FIG. 9 is a flowchart of a method 60 of attenuating electromagnetic radiation. The method 60 may specifically be employed in order to attenuate ultraviolet radiation. However, as explained herein, the device 10 is capable of attenuating radiation across the electromagnetic spectrum. Accordingly, the method 60 is not limited to the attenuation of ultraviolet radiation.

At 62, a device comprising a plurality of elongate flow passages is provided. The device may, for example, be the device 10 described with reference to FIGS. 1 to 6. Each of the plurality of elongate flow passages is configured to permit airflow through the elongate flow passage from an inlet of the device to an outlet of the device.

At 64, the device is positioned between a source of electromagnetic radiation and a particular location, thereby reducing the amount of electromagnetic radiation that reaches the particular location. For example, the source of electromagnetic radiation may be the ultraviolet lamps 28 of the air cleaning section 30 shown in FIG. 4, and the particular location may be the maintenance location 34 within the air handling system 24 shown in FIG. 4.

### Example

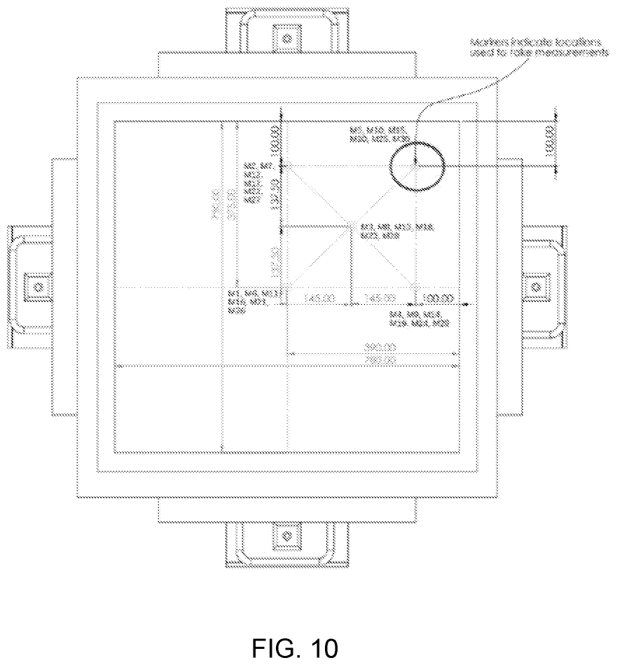

FIG. 10 shows locations of ultraviolet irradiance measurements in a system in which the device 10 was tested. The tested system included an air cleaning section in which ultraviolet lamps are housed in recesses around the periphery of the air cleaning section, such that the ultraviolet lamps are not disposed within the airflow through the air cleaning section (e.g. as schematically illustrated in FIG. 4). The ducting sections of the system have an interior width of 780 mm and an interior height of 750 mm. The ultraviolet irradiance within the air cleaning section is approximately 422 W/m².

The device for attenuating ultraviolet radiation used in the tests had a length of 120 mm and a honeycomb cell diameter of 9 mm. In other words, the aspect ratio of the device was 13.3.

A first series of measurement instances (M1, M6, M11, M21) is located at the centre of the ducting section cross-section. A second series of measurement instances (M2, M7, M12, M22) is located on a vertical centreline through the cross-section and 100 mm below the upper edge of the ducting section. A third series of measurement instances (M3, M8, M13, M23) is located at a midpoint between the second series of measurement locations and a point located on a horizontal centreline and 100 mm from a side wall of the ducting section (which is the location of a fourth series of measurement instances). In other words, the third series of measurements is located 137.5 mm above and 145 mm to the side of the centre of the cross-section. As noted above, the fourth series of measurement instances (M4, M9, M14, M24) is located on a horizontal centreline through the cross-section and 100 mm from a side wall of the ducting section. Finally, a fifth series of measurement instances (M5, M10, M15, M25) is located 100 mm below the upper edge of the ducting section and 100 mm from the side wall of the ducting section.

Measurement instances M1 to M5 were taken 20 mm from the outlet face of a device having elongate flow passages with internal walls that were uncoated (i.e. bare aluminium). Measurement instances M6 to M10 were taken 20 mm from the outlet face of a device having elongate flow passages with internal walls that were coated with a single coat of matte black paint. Measurement instances M11 to M15 were taken at the end of an additional duct that was fitted adjacent to the outlet face of the device used for taking measurements M6 to M10 (in order to replicate implementation in ducting of an air handling system). Measurement instances M21 to M25 were taken 20 mm from the outlet face of a device having elongate flow passages with internal walls that were coated with two coats of matte black paint.

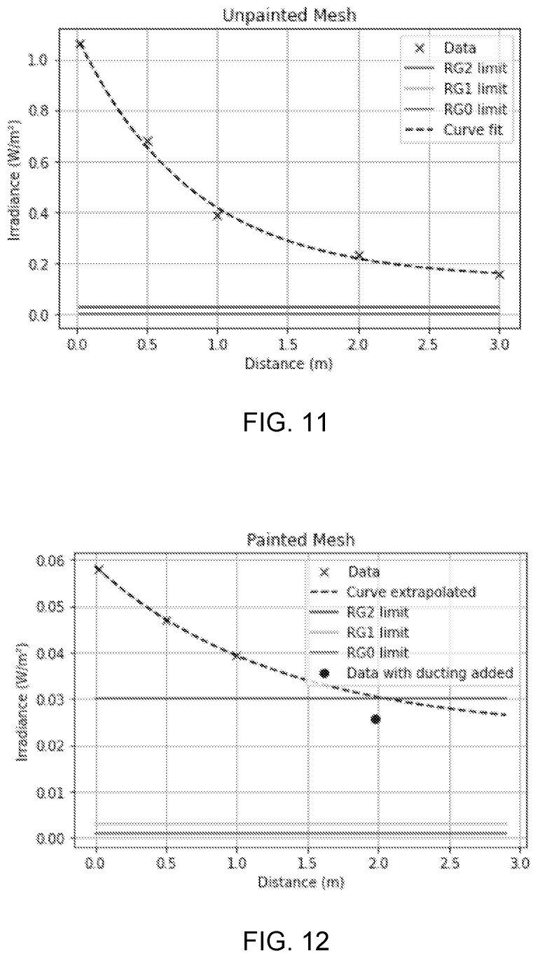

The fifth series of measurement instances is circled in FIG. 10 because irradiance values were highest at this series of instances. FIGS. 11 and 12 show ultraviolet irradiance values against distance for this point of maximum irradiance.

FIGS. 11 and 12 show ultraviolet irradiance values against the RG0, RG1 and RG2 limits defined in standards IEC 62471:2006 and BS EN 62471:2008 (Photobiological safety of lamps and lamp systems). The RG2 limit is 0.03 W/m² and can be seen in FIGS. 11 and 12 (i.e. the top horizontal line in both figures). The RG1 limit is 0.003 W/m² and can be seen in FIG. 12 (the middle horizontal line in FIG. 12). The RG1 limit cannot be seen in FIG. 11 as the RG0 limit line (which is of similar absolute value) is superimposed on the RG1 limit line. The RG0 limit is 0.001 W/m² and can be seen in FIGS. 11 and 12 (i.e. the bottom horizontal line in both figures).

FIG. 11 shows how the ultraviolet irradiance varies with distance from the outlet face of the device for measurement instance M5 (uncoated internal walls). The left-most data point in FIG. 11 shows the irradiance at the base location for measurement M5 (i.e. 20 mm from the outlet face), while the other data points identify irradiance values at different distances from the outlet face. As shown in FIG. 11, all data points are above the RG2 limit, with the left-most data point identifying an irradiance of 1.06 W/m².

FIG. 12 shows how the ultraviolet irradiance varies with distance from the outlet face of the device for measurement instance M10 (internal walls coated with a single coat of matte black paint). The left-most data point in FIG. 12 shows the irradiance at the base location for measurement M10 (i.e. 20 mm from the outlet face), while the other data points identify irradiance values at different distances from the outlet face. With no ducting fitted adjacent to the outlet face, the extrapolated curve shows that the RG2 limit is met at a distance of 2.1 m from the outlet face. The circular data point in FIG. 12 shows the effect of adding the ducting adjacent to the outer face, that is, a reduction below the RG2 limit at a distance of less than 2.1 m. This suggests that the RG2 limit is met at less than the 2.1 m distance when additional ducting is added.

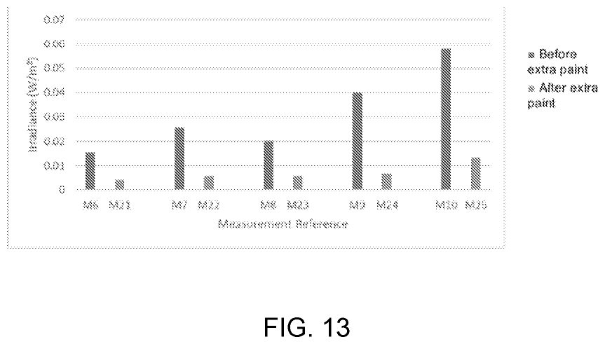

FIG. 13 is a comparison of measurement instances M6-M10 and M21-M25 (i.e. internal walls coated with a single coat of matte black paint compared with internal walls coated with two coats of matte black paint). The irradiance value for M10 (i.e. the point of maximum irradiance) is the same as the left-most data point in FIG. 12. FIG. 13 shows that the additional coat of matte black paint leads to a significant reduction in irradiance. In particular, at the location of the fifth series of measurement instances, the irradiance is reduced to 0.0135 W/m² for a device having elongate flow passages with internal walls that are coated with two coats of matte black paint. This value is below the RG2 limit.

Variations or modifications to the systems and methods described herein are set out in the following paragraphs.

Although the elongate flow channels 16 shown in FIGS. 1 and 3 have hexagonal cross-sections, it will be appreciated that incident ultraviolet radiation may be attenuated using other cross-sections of the elongate flow channels 16. For example, the elongate flow channels 16 may have circular, triangular, square, or rectangular cross-sections. In order to minimise the restriction to air flowing through the device 10, it is preferable if the elongate flow channels 16 have cross-sections that can be tessellated (such as triangles, squares, or hexagons).

In addition, although the ducting section 12 in FIGS. 1 to 7 has a square cross-section, it will be appreciated that the device 10 may be incorporated into a ducting section 12 having a different cross-section. For example, if a HVAC system includes circular ducts, then the device 10 may be incorporated into a ducting section 12 having a circular cross-section.

The skilled person will further appreciate that other colours of paint may be used to reduce the reflectivity of the internal walls 22 and/or to increase the diffuse nature of the reflection off the internal walls 22. For example, although black paint provides optimal absorbance of incident radiation, other dark-coloured paints will also provide reductions in reflectivity of incident radiation.

The above examples are described with reference to systems in which air flows through the ducting section. The skilled person will appreciate that the above examples are also applicable to systems used for handling gases other than air. In addition, although the above examples are described with particular reference to HVAC systems, the device 10 described herein may also be used in other settings in which leakage of ultraviolet photons is to be minimised without adversely impacting on air or gas flow.

It will also be appreciated that, although the above examples are described with reference to attenuating ultraviolet radiation (in particular, UVC radiation), the device 10 described herein may also be used to attenuate radiation across the electromagnetic spectrum. In other words, the capability of the device 10 to attenuate the energy of the radiation is independent of the wavelength of the radiation.

The singular terms “a” and “an” should not be taken to mean “one and only one”. Rather, they should be taken to mean “at least one” or “one or more” unless stated otherwise. The word “comprising” and its derivatives including “comprises” and “comprise” include each of the stated features, but does not exclude the inclusion of one or more further features.

The above implementations have been described by way of example only, and the described implementations are to be considered in all respects only as illustrative and not restrictive. It will be appreciated that variations of the described implementations may be made without departing from the scope of the invention. It will also be apparent that there are many variations that have not been described, but that fall within the scope of the appended claims.

1. A device for attenuating ultraviolet radiation in an air handling system, wherein the device is arranged for insertion in a ducting section of the air handling system, the device comprising:
a plurality of elongate flow passages, wherein each of the plurality of elongate flow passages is configured to permit airflow through the elongate flow passage from an inlet of the device to an outlet of the device;
wherein each of the elongate flow passages comprises one or more internal walls comprising a coating configured to absorb a proportion of incident ultraviolet radiation.

2. A device according to claim 1, wherein the device is arranged for insertion in the ducting section such that any airflow through the ducting section flows through the elongate flow passages of the device.

3. A device according to claim 1, wherein the plurality of elongate flow passages are arranged such that, collectively, the plurality of elongate flow passages has a substantially square or rectangular cross-section.

4. A device according to claim 1, wherein each of the plurality of elongate flow passages is configured to permit air to flow substantially unimpeded through the elongate flow passage from the inlet to the outlet.

5. A device according to claim 1, wherein each of the plurality of elongate flow passages is straight.

6. A device according to claim 1, wherein each of the plurality of elongate flow passages has an aspect ratio calculated by dividing a length of the elongate flow passage by a diameter of the elongate flow passage, and wherein the aspect ratio of each of the plurality of elongate flow passages is greater than or equal to 4.

7. (canceled)

8. A device according to claim 1, wherein the aspect ratio of each of the plurality of elongate flow passages is less than or equal to 20.

9. A device according to claim 1, wherein the coating is configured to absorb a proportion of incident UVC radiation.

10. (canceled)

11. A device according to claim 1, wherein the coating is configured to reflect less than 20% of incident UVC radiation.

12. A device according to claim 1, wherein the coating comprises one or more coats of black paint.

13. A device according to claim 1, wherein the device is for attenuating ultraviolet radiation in a heating, ventilation and air conditioning, HVAC, system comprising a source of ultraviolet radiation, and wherein the device is arranged for insertion in a ducting section of the HVAC system.

14. A ducting section comprising a device according to any of claims 1 to 13.

15. A ducting section according to claim 14, wherein the ducting section is for use in a heating, ventilation and air conditioning, HVAC, system comprising a source of ultraviolet radiation.

16-18. (canceled)

19. A system,
comprising: a ducting section according to claim 14; and
one or more baffles arranged between the plurality of elongate flow passages and an ultraviolet radiation source, wherein the one or more baffles are configured to prevent photons emitted by the ultraviolet radiation source from entering the plurality of elongate flow passages in a direction parallel to the plurality of elongate flow passages.

20. A heating, ventilation and air conditioning, HVAC, system comprising:
a source of ultraviolet radiation; and
a ducting section according to any of claims 14 to 18.

21. A method of manufacturing a device for attenuating ultraviolet radiation, the method comprising:
providing a device comprising a plurality of elongate flow passages, wherein each of the plurality of elongate flow passages is configured to permit airflow through the elongate flow passage from an inlet of the device to an outlet of the device; and
applying a first coat of a coating configured to absorb a proportion of incident ultraviolet radiation to one or more internal walls of each of the plurality of elongate flow passages.

22. A method according to claim 21, further comprising applying a second coat of the coating to the one or more internal walls of each of the plurality of elongate flow passages.

23. A method according to claim 21, wherein the coating comprises black paint.

24. A method of attenuating electromagnetic radiation, the method comprising:
providing a device comprising a plurality of elongate flow passages, wherein each of the plurality of elongate flow passages is configured to permit airflow through the elongate flow passage from an inlet of the device to an outlet of the device; and
positioning the device between a source of the electromagnetic radiation and a particular location, thereby reducing the amount of electromagnetic radiation that reaches the particular location.

25. A method according to claim 24, wherein the electromagnetic radiation is ultraviolet radiation.

26-29. (canceled)
